(19) United States
(12) Patent Application Publication
(10) Pub. No.: US 2005/0124988 A1
(43) Pub. Date: Jun. 9, 2005

(54) Modular navigated portal

(76) Inventors: Lauralan Terrill-Grisoni, Cordova, TN (US); Patrick Culley, Memphis, TN (US); Therise Ruffin, Collierville, TN (US); Kevin Raburn, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/959,283

(22) Filed: Oct. 5, 2004

Related U.S. Application Data
(60) Provisional application No. 60/509,366, filed on Oct. 6, 2003.

(51) Int. Cl. A61F 5/04; A61B 17/56

(52) U.S. Cl. 606/53

(57) ABSTRACT

A modular portal for use with a computer-aided surgical navigation system. The modular portal is configured to receive a wide number of surgical items in either rotating or non-rotating fashions. A surgical reference associated with a base of the modular portal may be repositioned with respect to the base to facilitate a clear line of sight between a sensor of the computer-aided surgical navigation system and the surgical reference.

This application relates to and claims the benefit on U.S. Provisional Application No. 60/509,366, filed Oct. 6, 2003 and entitled “Modular Navigated Portal,” the entire contents of which are hereby expressly incorporated by this reference.

### BACKGROUND

Many surgical procedures require a wide array of instrumentation and other surgical items. Necessary items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes and wrenches. In many surgical procedures, including orthopaedic procedures, it may be desirable to associate some or all of these items with a guide and/or handle incorporating a surgical reference, allowing the instrument to be used with a computer-aided surgical navigation system.

Several manufacturers currently produce computer-aided surgical navigation systems. The TREON™ and ION™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing computer-aided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; and U.S. Ser. No. 10/229,372, filed Aug. 27, 2002 and entitled “Image Computer Assisted Knee Arthroplasty”, the entire contents of each of which are incorporated herein by reference as are all documents incorporated by reference therein.

These systems and processes use position and/or orientation tracking sensors such as infrared sensors acting stereoscopically or other sensors acting in conjunction with surgical references to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated surgical references, or based on stored position and/or orientation information. The processing functionality correlates this position and orientation information for each object with stored information, such as a computerized fluoroscopic imaged filed, a wire frame data file for rendering a representation of an instrument component, trial prosthesis or actual prosthesis, or a computer generated file relating to a rotational axis or other virtual construct or reference. The processing functionality then displays position and orientation of these objects on a rendering functionality, such as a screen, monitor, or otherwise. Thus, these systems or processes, by sensing the position of surgical references, can display or otherwise output useful data relating to predicted or actual position and orientation of surgical instruments, body parts, surgically related items, implants, and virtual constructs for use in navigation, assessment, and otherwise performing surgery or other operations.

Some of the surgical references used in these systems may emit or reflect infrared light that is then detected by an infrared camera. The references may be sensed actively or passively by infrared, visual, sound, magnetic, electromagnetic, x-ray or any other desired technique. An active reference emits energy, and a passive reference merely reflects energy. Some surgical references may have at least three, but usually four, markers or fiducials that are traced by an infrared sensor to determine the position and orientation of the reference and thus the position and orientation of the associated instrument, item, implant component or other object to which the reference is attached.

In addition to surgical references with fixed fiducials, modular fiducials, which may be positioned independent of each other, may be used to reference points in the coordinate system. Modular fiducials may include reflective elements which may be tracked by two, sometimes more, sensors whose output may be processed in concert by associated processing functionality to geometrically calculate the position and orientation of the item to which the modular fiducial is attached. Like fixed fiducial surgical references, modular fiducials and the sensors need not be confined to the infrared spectrum—any electromagnetic, electrostatic, light, sound, radio frequency or other desired technique may be used. Similarly, modular fiducials may “actively” transmit reference information to a tracking system, as opposed to “passively” reflecting infrared or other forms of energy.

Surgical references useable with the above-identified navigation systems may be secured to any desired structure, including the above-mentioned surgical instruments and other items. The surgical references may be secured directly to the instrument or item to be referenced. However, in many instances it will not be practical or desirable to secure the surgical references to the instrument or other item. Rather, in many circumstances it will be preferred to secure the surgical references to a handle and/or a guide adapted to receive the instrument or other item. For example, drill bits and other rotating instruments cannot be tracked by securing the surgical reference directly to the rotating instrument because the reference would rotate along with the instrument. Rather, a preferred method for tracking a rotating instrument is to associate the surgical reference with the instrument or item's guide or handle.

Typical guides and handles are adapted to each only support one type of instrument or item. Consequently, a guide or handle must be provided for each instrument and item to be used in the surgical procedure. This may be undesirable for several reasons. Using a unique guide or handle for each surgical item substantially increases the amount of instrumentation present in the operating room. Additionally, it requires removing and installing new guides when different instruments are to be used, increasing the time and risk of infection associated with the surgical procedure.

### SUMMARY

Various aspects and embodiments of the present invention include modular portals adapted to serve as guides or handles for a wide variety of surgical instruments and other items. The modular portals may be adapted to allow the surgical items to access interior portions of an individual. For instance, modular portals according to certain embodiments of the present invention may be used to obtain unobstructed access to desired bone structures in an individual for performance of surgical procedures, such as installation of an implant. Additionally, modular portals according to certain embodiments of the present invention may allow the surgical instruments and other items received by the modular portals to be registered in and tracked by a computer-aided surgical navigation system. Such systems may track the position and orientation of the surgical item by tracking the position and orientation of the surgical reference associated with the modular portal. Because the modular portals may be used with a wide range of surgical items for a wide range of purposes, a unique guide and/or handle does not have to be constructed for each surgical item used in any given procedure.

Modular portals according to certain aspects and embodiments of the present invention may include a base and a handle. The handle may or may not be integral to the base. Modular portals may also be associated with a surgical reference such that the position and orientation of the modular portal, as well as instruments or items associated with the modular portal, may be tracked by an image guided surgical-navigation system. In certain embodiments, the surgical reference may be associated with the base. In some of these embodiments, the base may be adapted to allow the surgical reference to be repositioned with respect to the base such that the surgical reference is in a convenient location. In other embodiments where the surgical reference is not associated with the base, the surgical reference may be secured to a platform that also supports the base, or other portions of the modular portal.

Bases according to certain aspects and embodiments of the present invention may include apertures or other appropriate structure allowing the modular portals to receive surgical instruments and other items of various shapes and sizes. The instruments or other items receivable by the aperture may include a number of sleeves having different lengths and diameters. The sleeves may be secured to the base in any desired manner. In one embodiment, threads on proximal ends of the sleeves interact with corresponding threads in the aperture to secure the sleeves to the modular portal.

In some embodiments, tips of various sizes and shapes may be interchangeably secured onto distal ends of the sleeves. The tips may be formed in a variety of configurations. For instance, a tip may be formed such that when the tip contacts a particular bone structure, the tip assists in centering the sleeve on that bone structure. Some tips may include teeth to further stabilize the modular portal (including the sleeve) with respect to the bone. Still other tips may be adapted to perform other desired functions. In some embodiments, the sleeves may include integrally formed tips. In other embodiments, the tips may be secured to the sleeves by any appropriate and/or desired structure.

Sleeves according to various aspects and embodiments of the present invention may be used for a variety of functions and purposes. Sleeves according to certain embodiments may be used as entry tools, drill guides, tissue protectors and/or working channels. In some embodiments, two or more sleeves may be used simultaneously with a modular portal. For example, in one embodiment, a sleeve with a larger diameter may be used as a working channel and a sleeve with a smaller diameter may be mounted inside the working channel for use as a drill guide.

Other surgical instruments or items may be used and navigated with modular portals in accordance with certain embodiments of the present invention with or without sleeves. The surgical items may be secured, received and/or supported by the modular portal in any desired manner. In some embodiments, other surgical items may be secured to the modular portal in the same manner as the sleeves described above. In some embodiments, the modular portal may include retaining mechanisms, such as a number of spring plungers that interact with a channel or channels on the surgical item such that the item is secured to the modular portal yet able to rotate.

Surgical instruments and other items receivable by modular portals of this invention may include axisymmetric or non-axisymmetric items. In some embodiments, non-axisymmetric instruments or items may be locked into a certain configuration with regard to the modular portal through the addition of a key and a keyway.

In some embodiments, the modular portal may include a measuring tool. The measuring tool may measure the length and/or diameter of an instrument or other item passing through the portal such that a correct graphical representation of the length and/or width of the instrument inserted through the portal can be displayed by the computer-aided surgical navigation system. Use of such a measuring tool may obviate the need to recalibrate the computer-aided surgical navigation system when different instruments or items are used. The measuring tool may be mechanical (such as a rotating wheel), electronic, electromagnetic or optical (such as a laser).

In some embodiments, the modular portal may be used to reduce or eliminate the possibility of airborne contamination of the surgical wound. In such embodiments, the modular portal may include a suction tube to provide suction through the modular portal such that any blood associated with the wound is not aerosolized.

In some embodiments, interface buttons communicatively associated with the computer-aided surgical navigation system or other computer functionality may be included on the handle, or other appropriate structure, of the modular portal. The interface buttons may communicate with the image-guided surgical navigation structure or other computer functionality using either wireless or wired technology. The interface buttons may function in lieu of or in addition to a conventional footswitch, mouse, keyboard or touch-screen buttons associated with the computer-aided surgical navigation system or other computer functionality.

### BRIEF DESCRIPTION OF THE DRAWINGS

FIG. 1 shows a modular portal according to a first embodiment of the present invention in perspective view.

FIG. 2 shows a schematic view of a modular portal according to another embodiment of the present invention including a suction tube.

FIG. 3 shows a modular portal according to another embodiment of the present invention in perspective view including a single interface button.

FIG. 4 shows a modular portal according to another embodiment of the present invention in perspective view including two interface buttons.

FIG. 5 shows a modular portal according to another embodiment of the present invention in perspective view including a sleeve secured to the modular portal.

FIG. 6 shows a modular portal according to another embodiment of the present invention in perspective view including two sleeves secured to the modular portal.

FIG. 7 shows a modular portal according to another embodiment of the present invention in perspective view including a sleeve with an attached beveled self-centering tip.

FIG. 8 shows the sleeve of FIG. 7 disconnected from the modular portal in perspective view.

FIG. 9 shows a modular portal according to another embodiment of the present invention in perspective view including a short drill guide sleeve.

FIG. 10 shows a modular portal according to another embodiment of the present invention in perspective view including a long drill guide sleeve.

FIG. 11 shows a modular portal according to another embodiment of the present invention in perspective view including an osteotome blade.

FIG. 12 shows a modular portal according to another embodiment of the present invention in perspective view including a honeycomb drill guide.

FIG. 13 shows a modular portal according to another embodiment of the present invention in schematic view including a reducer/distractor.

FIG. 14 shows the reducer of FIG. 13 with its portal securing mechanism in an exploded view.

FIG. 15 shows a computer-aided surgical navigation system according to another embodiment of the present invention.

### DETAILED DESCRIPTION

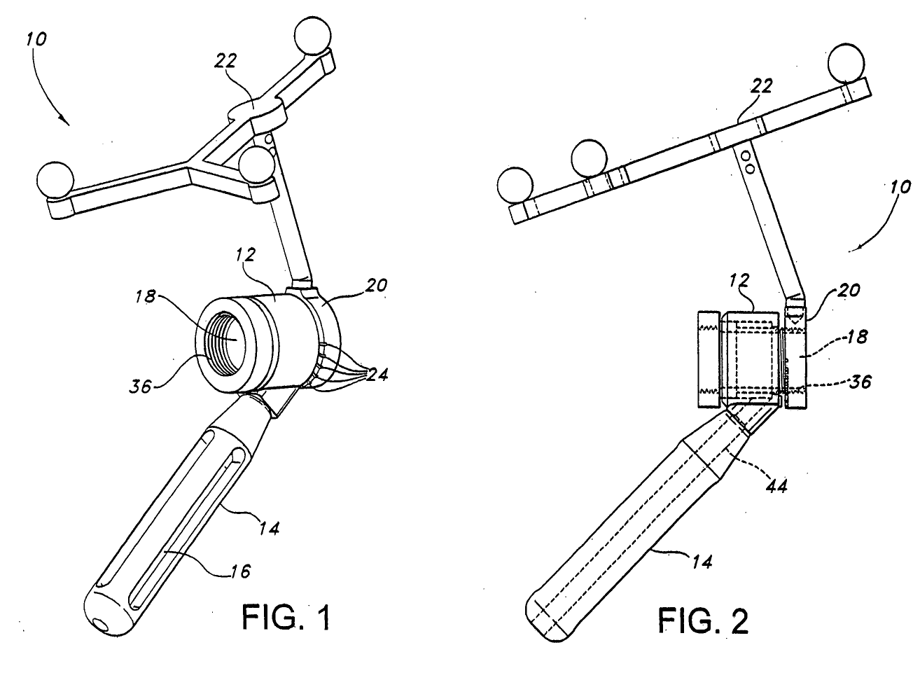

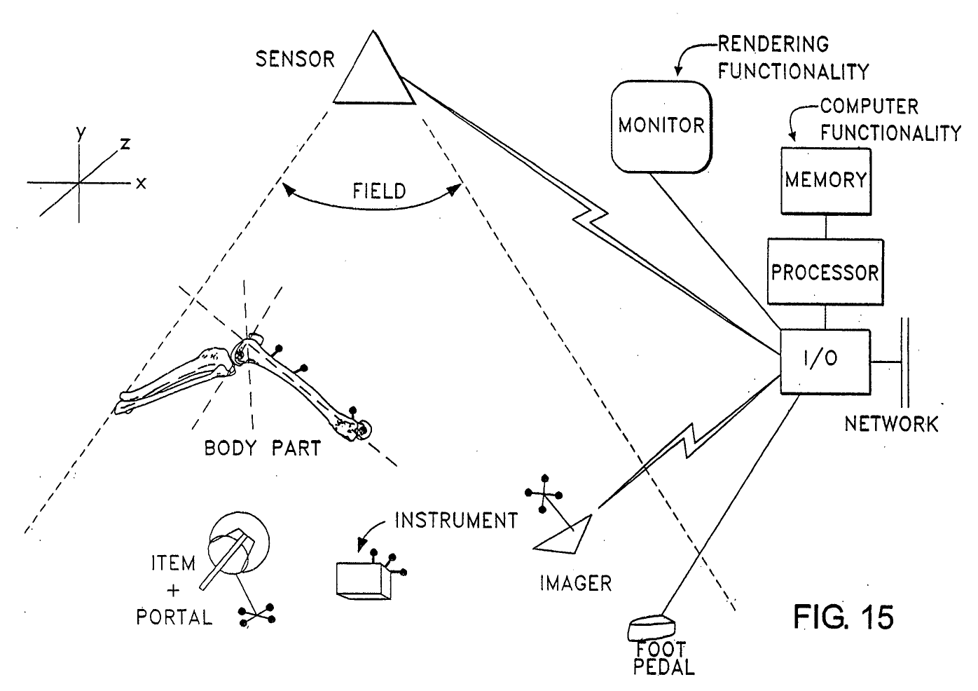

FIG. 1 shows a modular portal 10 according to a first embodiment of the present invention. Modular portals 10 may be used as handles and/or guides for a wide array of surgical instruments or other surgical items. In some embodiments, such as the embodiment shown in FIG. 1, modular portal 10 may be associated with a surgical reference 22. Surgical reference 22 may allow modular portal 10, as well as the surgical instruments or items received by modular portal 10, to be tracked by a computer-aided surgical navigation system, such as the system shown in FIG. 15. In some embodiments, modular portal 10 may be used in conjunction with certain surgical instruments and/or items to provided unobstructed access to interior portions of an individual.

The modular portal 10 shown in FIG. 1 includes a base 12 and a handle 14. In some embodiments, handle 14 may be formed integrally with base 12. In other embodiments, handle 14 may be connected to base 12 by any desired and/or appropriate structure. Preferably, the handle 14 is connected to the base 12 by threaded portions on the handle 14 and base 12. Handle 14 may be a typical instrument handle used in operating rooms. However, handle 14 may be formed in any desired shape and/or size. In the embodiment shown in FIG. 1, handle 14 includes a number of slots 16. Slots 16 may make it easier for a surgeon to grip the handle and/or may allow the handle to be mounted to other desired structure.

In the embodiment shown in FIG. 1, base 12 is secured to a surgical reference support member 20 that allows a surgical reference 22 to be rigidly mounted to modular portal 10. In some embodiments, support 20 may be repositioned with respect to base 12 such that the surgical reference 22 can be repositioned with respect to the modular portal 10. In the embodiment shown in FIG. 1, a pin (not shown) on the base 12 engages one of a plurality of notches 24 in the support 20 to orient the support 20 with respect to the base 12. Disengagement of the pin from the notch 24 allows the support 20 to be repositioned with respect to the base 12. Once the support 20 is oriented in a desired position, the pin can be inserted into a corresponding notch 24, securing the support 20 in a desired orientation with respect to the base 12. Adjusting the position of surgical reference 22 in this manner may allow the surgeon or other user to orient the surgical reference 22 in a convenient location and/or in a location that can be better visualized by the computer-aided surgical navigation system.

Suitable structures other than pins and notches 24 may be used to orient surgical reference 22 with respect to modular portal 10. In other embodiments, support 20 is fixed with respect to base 12 and cannot be repositioned. In still other embodiments, support 20 is not necessary. Rather, surgical reference 22 may be associated with modular portal 10 in any suitable and/or acceptable manner. For instance, surgical reference 22 may be secured to other portions of modular portal 10 or may be secured to other structures associated with modular portal 10. In one embodiment, surgical reference 22 is secured to a platform that supports and stabilizes the modular portal 10 with respect to the individual being operated on.

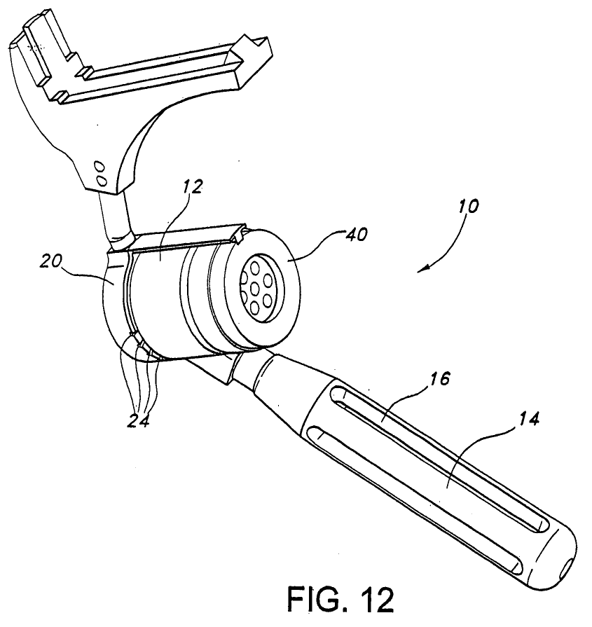

Surgical reference 22 may be any structure that can be tracked by a computer-aided surgical navigation system. For instance, as illustrated in FIG. 1, the surgical reference 22 may be a conventional reference structure. In other embodiments, surgical reference 22 may be a number of modular fiducials. In still other embodiments, surgical reference 22 is a reference transmitter or receiver. FIG. 12 shows support 20 adapted to support and secure an electromagnetic reference receiver.

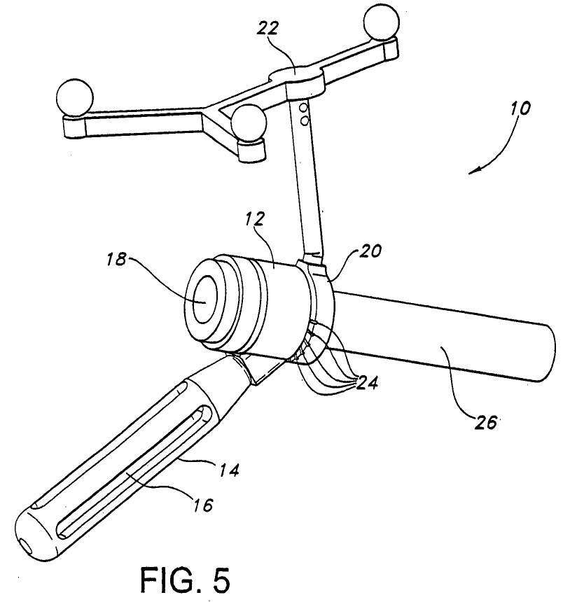

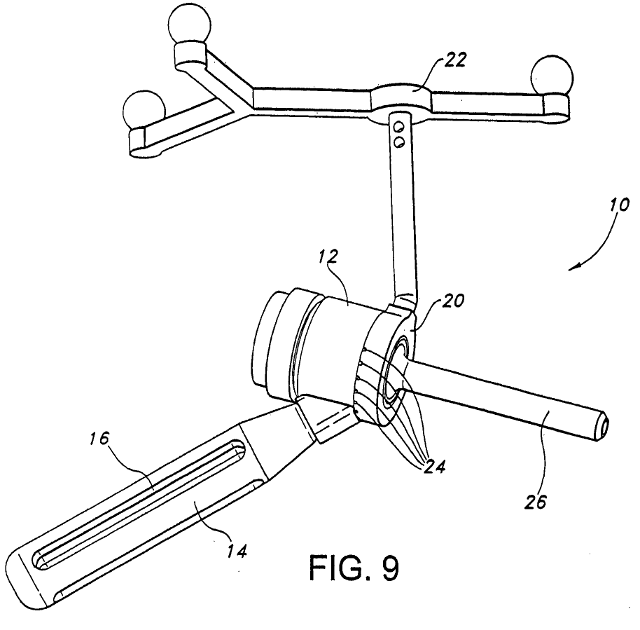

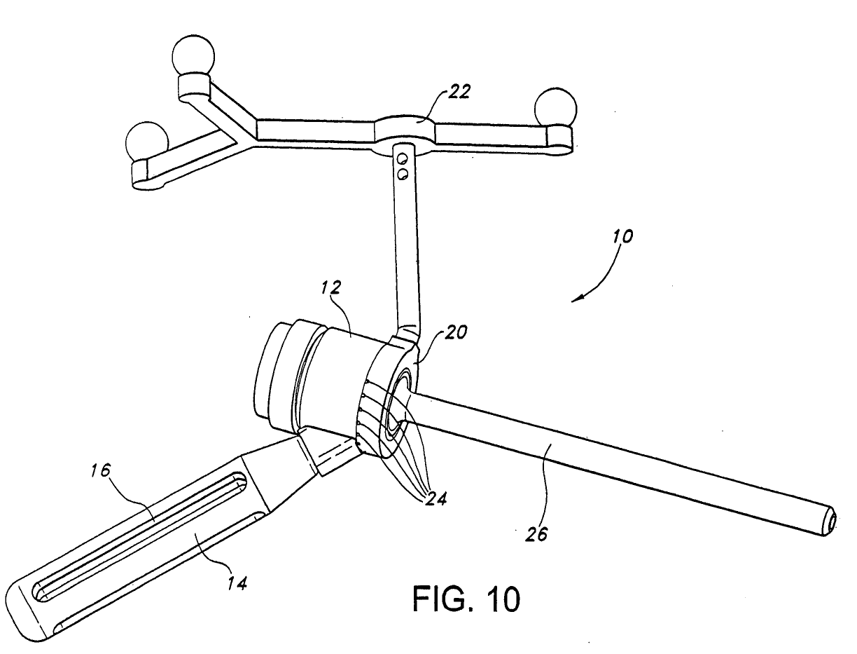

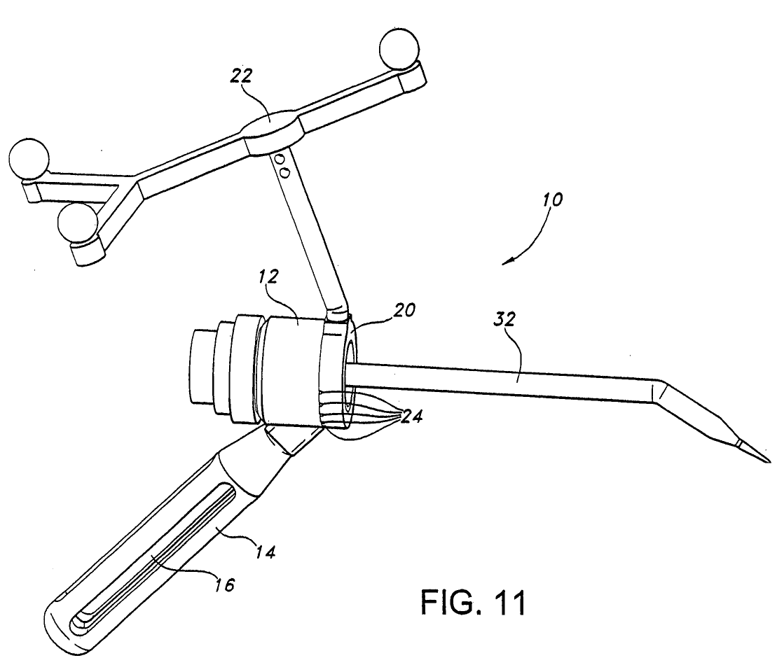

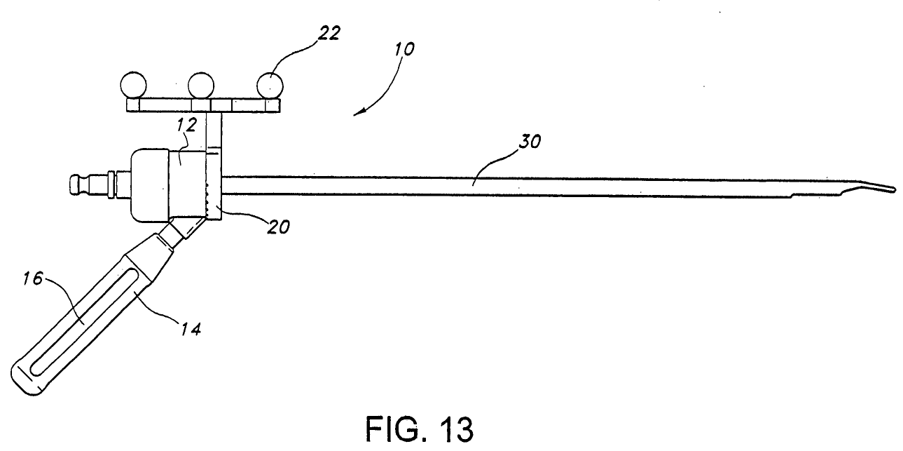

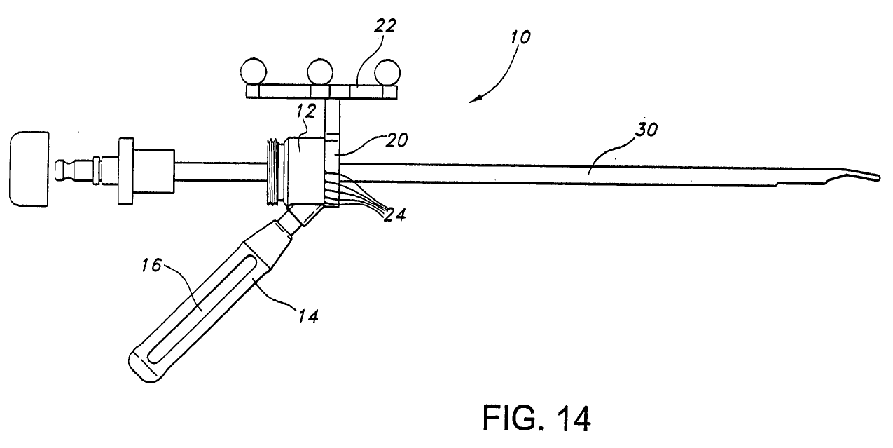

Base 12 may include an aperture 18 extending through a longitudinal portion of the base 12. Aperture 18 may be of a suitable diameter to allow a wide variety of items to be passed through aperture 18 and/or secured to modular portal 10. For instance, aperture 18 may be adapted to receive sleeves 26 of various shapes and sizes. Sleeves 26 may serve as working channels (such as the working channel shown in FIG. 5); drill sleeves (such as the drill sleeves shown in FIGS. 9 and 10); entry tools; eccentric sleeves; tissue protectors and/or any other desired functions. Aperture 18 may also receive other surgical instruments, such as the reducers 30 shown in FIGS. 13 and 14; the osteotome blade 32 shown in FIG. 11; the honeycomb drill sleeve 34 shown in FIG. 12; drill bits; wrenches; screwdrivers, awls, guide wires, guide rods or any other desired instrument. Aperture 18 may also allow other items, such as surgical implants (including intramedullary nails), to pass through it.

According to certain embodiments, because modular portal may be associated with a surgical reference 22, the position and/or orientation of which can be tracked by a computer-aided surgical navigation system, aperture 18 may be used as a navigated target for a variety of purposes. For example, aperture 18 may be used as a working channel to navigate the entry point for a surgical incision. Aperture 18 could also act as a navigated target entry zone indicator for passing through a scope (such as an endoscope or an arthroscope) to allow visualization and identification of structures inside the patient, such as soft tissue structures.

In certain embodiments, modular portals 10, as navigated handles or guides, may be used with surgical instruments or other items for many purposes. For instance, modular portals 10, in conjunction with appropriate instruments or other items such as sleeves 26, could act as navigated tissue protectors. Modular portals 10 in conjunction with appropriate sleeves 26, could navigate an entry point into a patient's bone by aiding in the navigation of an entry awl or guide pin. Additionally, modular portals 10 may be used to aid in the preparation of bones to receive implants by navigating reamer diameters and depths. Similarly, modular portals 10, with or without sleeves 26, may be used to navigate reducers, distractors and/or guide rods to aid in performing fracture reductions. Additionally, modular portals 10 may be used to navigate insertion and/or placement of implants (such as an intramedullary nail).

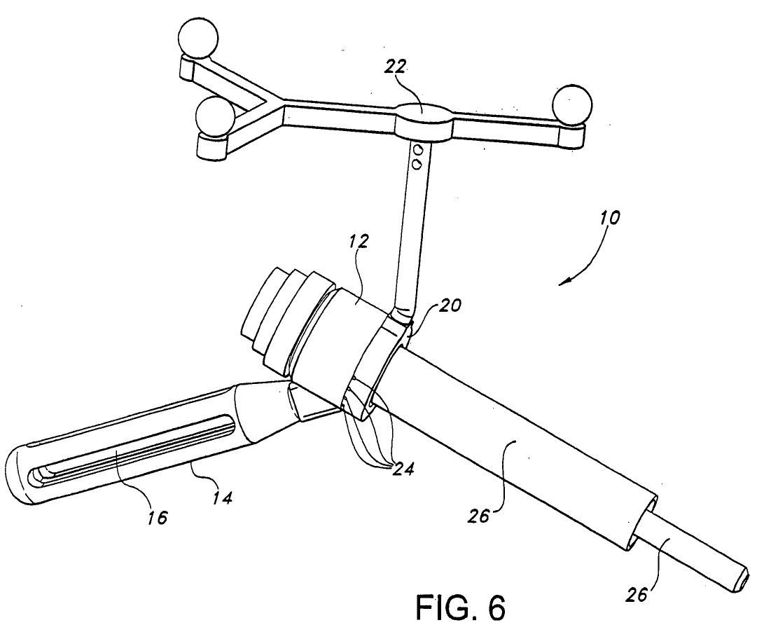

Instruments and other items useable with modular portals 10 according to certain embodiments of the present invention may be axisymmetric (such as the sleeves 26 shown in FIGS. 5-10) or non-axisymmetric (such as the osteotome blade 32 shown in FIG. 11 and the reducers 30 shown in FIGS. 13 and 14). In some embodiments, aperture 18, or another suitable portion of modular portal 10, may include either a key or a keyway to correspond to a keyway or a key located on a non-axisymmetric instrument to orient the instrument with respect to the modular portal in a defined geometry. Because, in certain embodiments, modular portals 10 and the associated surgical references 22 are fixed in defined geometries with respect to the non-axisymmetric instruments, the position and/or orientation of the non-axisymmetric instruments can be tracked by a computer-aided surgical navigation system.

Surgical instruments and items useable with modular portals 10 may be secured to and/or guided by modular portals 10 in any desired manner. For instance, in the embodiment shown in FIG. 1, threads 36 circumscribing a portion of the interior surface of the aperture 18 may interact with appropriately sized and shaped threads on an instrument or item to secure the instrument or item to the modular portal 10. FIGS. 5-7 and 9-10 show various sleeves secured to the modular portals 10. FIG. 11 shows an osteotome blade 32 secured to a modular portal 10. FIG. 12 shows a honeycomb drill guide 40 secured to a modular portal 10, which may be used to navigate multiple pins simultaneously. In some embodiments, a bracket on the honeycomb drill guide 40 secures the honeycomb drill guide 40 to the modular portal 10 in such a way that the honeycomb drill guide 40 rotates with the associated surgical reference 22. FIGS. 13 and 14 show reducers/distractors 30 in an assembled and an exploded view respectively secured to the modular portal 10. Reducers/distractors 30 may be used for bone segment manipulation or reduction. As illustrated by FIG. 14, threads do not have to be located on an interior surface of aperture 18. In the embodiment shown in FIGS. 13 and 14, threads of the outside of base 12 interact with threads on a portion of reducer 30 to secure reducer 30 to the modular portal 10. Other surgical instruments and other items may be secured to modular portal 10 simply by adding appropriately sized and shaped threads to the item.

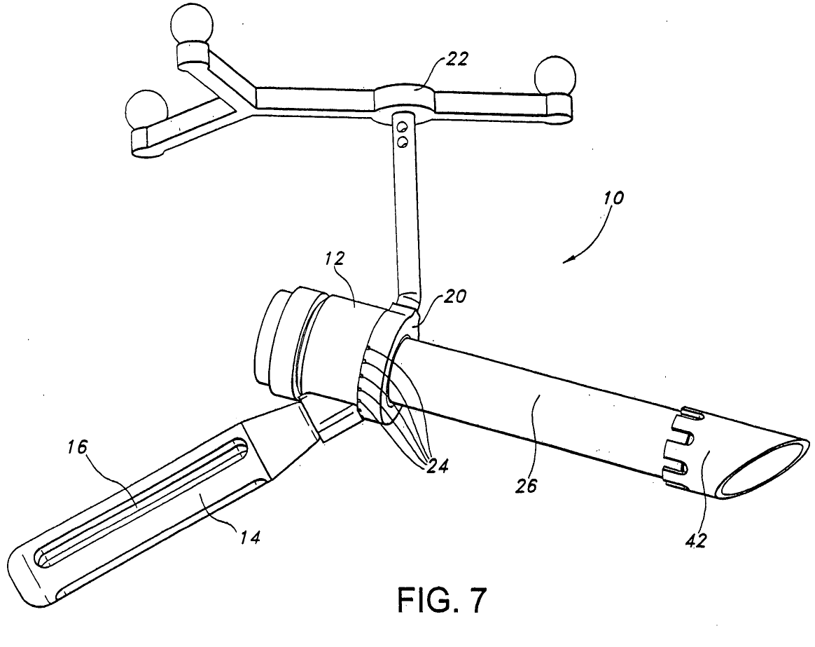

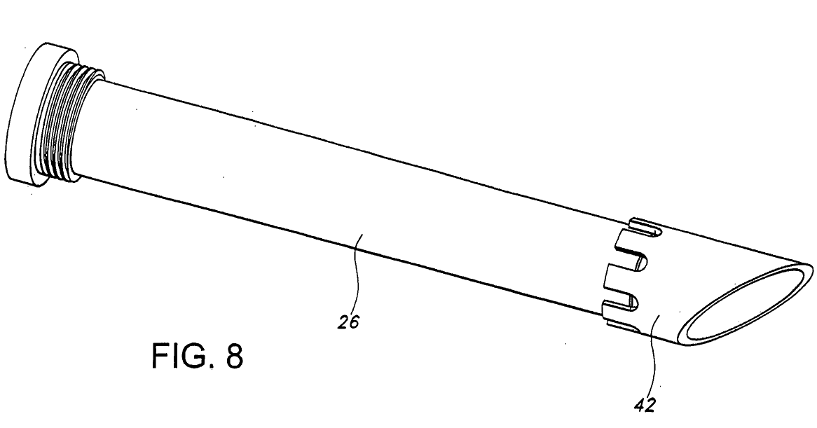

In the embodiment shown in FIGS. 7 and 8, the sleeve 26 includes a tip 42 secured to a distal end of the sleeve 26. Tips 42 may be secured to sleeves 26 in any desired manner. In the embodiment shown in FIGS. 7 and 8, tip 42 is secured to the sleeve 26 by snap locks on the tip 42. The snap locks may lock into a channel, ridge or any other appropriate structure on the sleeve 26. In other embodiments, the snap locks, or other appropriate structures, may be located on the sleeve; and the ridge or other appropriate structure may be located on the tip 42.

Tips 42 may be formed in a variety of shapes and sizes for a variety of purposes. In the embodiment shown in FIGS. 7 and 8, tip 42 is beveled, increasing the stability of the sleeve 26 when the tip 42 is resting on certain bone structures. The beveled tip 42 may also allow the tip 42 to self-orient itself on the bone so that the desired sleeve and/or instrument orientation is automatically achieved. In other embodiments, the tip 42 may be formed in other desired geometries to assist positioning the sleeve 26 with respect to a desired bone. In some embodiments, the tip 42 may be formed with or without teeth. The teeth may further stabilize the sleeve with respect to the bone. However, because teeth may cause skiving off bones in certain circumstances, they are not desirable in every situation. Consequently, other tips 42 may be formed without teeth. In some embodiments, some or all of these tips 42 may be formed integrally with desired and/or appropriate sleeves 26.

Surgical Instruments and other items may be secured to and/or guided by modular portal 10 in a rotating or non-rotating manner. These surgical items may be secured to and/or guided by modular portal 10 in conventional or non-conventional manners. In some embodiments, retaining mechanisms may extend from interior portions of aperture 18. The retaining mechanisms may be adapted to interact with appropriately shaped structures on the desired instrument or other item such that the item is secured to the modular portal 10, yet able to rotate with respect to the modular portal 10. In a preferred embodiment, retaining mechanisms are rings of spring plungers extending around an inner circumference of aperture 18. In the preferred embodiment, the instrument or other item includes at least one channel extending around a portion of the item such that insertion into the aperture 18 causes spring plungers to engage the channel, securing the item to the modular portal 10 in a rotating manner.

In some embodiments, the modular portal 10 may include a measuring tool. The measuring tool may be adapted to measure the length and/or diameter of certain instruments or other items passing through aperture 18 of the modular portal 10. Measuring the length and/or diameter of the item passing through aperture 18 may allow the item to be properly displayed by the computer-aided surgical navigation system without requiring a surgical reference secured directly to the item. The measuring device may be any appropriate device for measuring length and/or width, including mechanical, electrical and/or optical devices. In one embodiment, the measuring device may include a wheel that is adapted to contact a portion of the instrument or item passing through the aperture 18. As the instrument or item moves through the aperture 18, the wheel turns. The wheel may be associated with computer functionality such that the computer functionality can calculate the length of the portion of instrument that has passed through aperture 18 based on how much the wheel has turned. Additionally, the computer functionality may relay this information to the computer-aided surgical navigation system such that the proper position and/or orientation of the instrument is displayed.

In some embodiments, such as the embodiment shown in FIG. 2, the modular portal 10 may include suction functionality. In such embodiments, the modular portal 10 may be adapted to connect to typical suction tubes used in operating rooms. In the embodiment shown in FIG. 2, the handle 14 is adapted to receive a suction tube, such as the suction tubes commonly used in operating rooms. A suction pipe 44 extending through the handle 14 into the aperture 18 allows suction to be applied into the aperture 18. The application of suction into the aperture 18, and consequently into the surgical wound, may reduce the amount of aerosolized blood present in the wound. Reducing the amount of aerosolized blood may reduce the chances of infection and contamination.

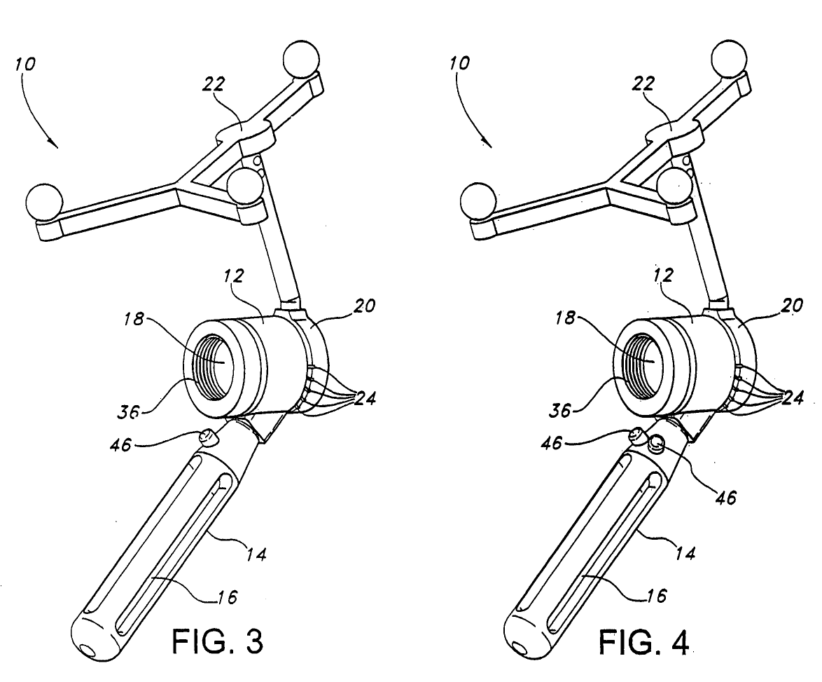

In some embodiments, such as the embodiments shown in FIGS. 3 and 4, the modular portal 10 may include one or more interface buttons 46. The interface button 46 may be connected to computer functionality, including the computer-aided surgical navigation system, using wired or wireless technology. The interface button 46 may allow the surgeon or other user to interface with the computer functionality without removing his or her hands from the handle 14 or other portions of the modular portal 10. Interface buttons 46 according to certain aspects and embodiments of the present invention may be used in much the same way as typical interface buttons used in surgical settings, including footswitches, mice and touch-screen buttons.

In some embodiments, the modular nature of the portal 10 allows the above described items as well as other surgical instruments and/or items to be used in conjunction with the modular portal 10 without removing the modular portal 10 from the patient. Rather, an instrument, sleeve 26, or other item associated with the modular portal 10 could be removed such that another desired instrument, sleeve 26 or item can be associated with the portal 10. The removal and replacement can all be accomplished without removing the portal 10 (and other items associated with the portal 10) from the patient, increasing the speed of the surgical procedure and lessening the chance of infection to the patient. Also, the modular nature of portal 10 allows a number of instruments and other items to be used in conjunction with a computer-aided surgical navigation system without creating a unique handle/grip or substantially modifying the surgical items.

Modular portals 10 according to various aspects and embodiments of the present invention may be used for a wide variety of surgical procedures. For instance, during the installation of an intramedullary nail into a femur, the modular portal 10 may serve a number of functions. Initially, the aperture 18 of the modular portal 10 could be used as a navigated working channel for locating the proper incision area. Subsequently, the aperture 18 could act as a navigated target entry zone indicator for passing a scope into the incision to visualize soft tissues and other structures. Subsequently, a sleeve 26 could be installed into the modular portal 10, creating a working channel for passing through other instruments necessary to complete the surgical procedure. The sleeve 26 could also serve the purpose of protecting soft tissues and other internal structures from damage during the surgical procedure. A beveled tip 42 with teeth could be included on a distal end of the sleeve to aid in locating the sleeve proximate the proper entry point into the intramedullary canal. An entry awl, guide pin, or other suitable instrument could then be passed through the working channel such that the instrument is navigated to the proper entry point into the bone. After the entry point is identified, the modular portal could be used to navigate a reamer diameter and depth to prepare the bone to receive the intramedullary nail. If necessary or desired, the modular portal could also be used to navigate a reducer, distractor and/or guide rod through the working channel to perform a fracture reduction. The modular portal may also be used to navigate the nail implant through the portal 10. All of these steps may be performed without removing the modular portal 10 from the patient's soft tissue.

The same modular portal 10 may be used to distally lock the intramedullary nail by replacing the long sleeve 26 including the beveled tip 42 with teeth with a shorter sleeve 26 with a tip 42 that does not include teeth. Teeth are generally not preferred for performing distal locking because the teeth may cause skiving of the sleeve off of the bone during drilling.

Modular portals 10, according to certain embodiments of the present invention, may also be used (with or without sleeve 26) in other surgical procedures. Other uses include: navigation of endoscopes or arthroscopes; navigation of entry points and positioning of instruments and implants; navigation of osteotomes 32 to aid in resectioning bone; navigation of cutting tools such as saws, reamers, and drill bits; navigation of sleeves and injectors for the placement of biologic agents, therapeutic agents and bone cement; navigation of dowel and plug bone cutting and grafting procedures; and navigation of bone stimulation therapies. Consequently, modular portals 10 according to certain aspects and embodiments of the present invention provide a versatile tool that can not only be used as a simple drill guide, but also as a navigated handle for a wide variety of applications.

Changes and modifications, additions and deletions may be made to the structures recited above and shown in the drawings without departing from the scope or spirit of the invention and the following claims.

1. A modular portal for use with a computer-aided surgical navigation system, the system comprising: a sensor, which senses the position and orientation of a surgical reference associated with the modular portal; computer functionality, which receives information from the sensor about position and orientation of the surgical reference and generates information corresponding to position and orientation of a surgical item; and rendering functionality, which renders at least the position and orientation of the surgical item in correspondence with the position and orientation of the surgical reference as sensed by the sensor; the modular portal comprising:
(a) a base comprising an aperture extending through the base;
(b) a surgical reference associated with the base;
(c) a first surgical item securable to the base; and
(d) a second surgical item securable to the base
wherein at least one of the first surgical item and second surgical item are secured to the base and wherein the surgical item secured to the base extends through the aperture of the base.

2. The modular portal of claim 1, wherein the first surgical item is able to be rotated with respect to the base when the first surgical item is secured to the base.

3. The modular portal of claim 1, wherein the first surgical item is not able to be rotated with respect to the base when the first surgical item is secured to the base.

4. The modular portal of claim 1, wherein the first surgical item defines an aperture.

5. The modular portal of claim 4, wherein the first surgical item comprises a sleeve.

6. The modular portal of claim 1, further comprising suction functionality.

7. The modular portal of claim 6, further comprising a handle extending from the base; and wherein the handle further comprises a suction tube.

8. The modular portal of claim 1, further comprising at least one interface button.

9. The modular portal of claim 8, further comprising a handle extending from the base; and wherein the at least one interface button is located on the handle.

10. The modular portal of claim 1, wherein the surgical reference is able to be repositioned with respect to the base.

11. The modular portal of claim 10, further comprising a support member; wherein the support member is secured to the base; wherein the support member supports the surgical reference; and wherein the support member is able to be repositioned with respect to the base.

12. The modular portal of claim 11, wherein the support member is able to be repositioned with respect to the base in one of a plurality of discrete orientations.

13. The modular portal of claim 12, wherein a plurality of notches in the support member determine the plurality of discrete orientations.

14. A method for performing a surgical procedure using a modular portal and a computer-aided surgical navigation system, the system comprising: a sensor, which senses the position and orientation of a surgical reference associated with the modular portal; computer functionality, which receives information from the sensor about position and orientation of the surgical reference and generates information corresponding to position and orientation of a surgical item; and rendering functionality, which renders at least the position and orientation of the surgical item in correspondence with the position and orientation of the surgical reference as sensed by the sensor; the method comprising:
(a) assembling a modular portal, wherein the modular portal comprises:
(i) a base comprising an aperture extending through the base;
(ii) a surgical reference associated with the base;
(iii) a first surgical item securable to the base; and
(iv) a second surgical item securable to the base
wherein assembling the modular portal comprises securing at least one of the first surgical item and second surgical item to the base and wherein the surgical item secured to the base extends through the aperture of the base;
(b) using the first surgical item to perform a first surgical procedure;
(c) securing a second surgical item to the base;
(d) using the second surgical item to perform a second surgical procedure.

15. The method for performing a surgical procedure of claim 14, further comprising using the modular portal to provide suction.

16. The method for performing a surgical procedure of claim 14, further comprising repositioning the surgical reference with respect to the base.

17. The method for performing a surgical procedure of claim 16, wherein repositioning the surgical reference with respect to the base comprises rotating a support member with respect to the base.

18. The method for performing a surgical procedure of claim 14, further comprising using an interface button on the modular portal to communicate with the computer-aided surgical navigation system.

19. The method for performing a surgical procedure of claim 14, further comprising installing an implant.

20. The method for performing a surgical procedure of claim 19, wherein installing an implant is installing a intramedullary nail, installing a hip implant, installing a knee implant or installing a shoulder implant.
